(19) United States
(12) Patent Application Publication
(10) Pub. No.: US 2026/0311351 A1
(43) Pub. Date: Oct. 8, 2026

(54) SYSTEM AND METHOD FOR VESSEL-BASED DEFORMABLE ALIGNMENT IN LAPAROSCOPIC GUIDANCE

(71) Applicant: EDDA TECHNOLOGY, INC., PRINCETON, NJ (US)

(72) Inventors: Xiaonan Zang, Princeton, NJ (US); Guo-Qing Wei, Plainsboro, NJ (US); Li Fan, Belle Mead, NJ (US); Xiaolan Zeng, Princeton, NJ (US); Jianzhong Qian, Princeton Junction, NJ (US)

(21) Appl. No.: 19/479,028

(22) PCT Filed: Apr. 26, 2024
(86) PCT No.: PCT/US2024/026557

§ 371 Date: Oct. 27, 2025

Related U.S. Application Data
(60) Provisional application No. 63/498,699, filed on Apr. 27, 2023.

(51) Int. Cl. G06T 7/33; A61B 34/20; A61B 90/00; G06T 7/73

(52) U.S. Cl. CPC G06T 7/344; A61B 34/20; A61B 90/37; G06T 7/73; A61B 2034/2057; G06T 2207/30101

(57) ABSTRACT

The present teaching relates to method, system, and medium for deformable alignment. 2D vessels are automatically detected from 2D images having anatomical structures captured by a camera inserted into a patient's body and directed to a target organ. A vessel label is obtained to at least one 2D vessel. A 3D vessel structure in a 3D model for the target organ is identified based on the detected 2D vessels. The 2D vessels are aligned with 3D vessels in the 3D vessel structure so that alignment parameters for each pair may be derived and used for visualizing 3D vessels corresponding to the detected 2D vessels.

### CROSS REFERENCE TO RELATED APPLICATION

The present application claims priority benefit of the filing date of U.S. Provisional Patent Application No. 63/498,699, filed Apr. 27, 2023, which is herein incorporated by reference in its entirety.

### BACKGROUND

### 1. Technical Field

The present teaching generally relates to computers. More specifically, the present teaching relates to signal processing.

### 2. Technical Background

With the advancement of technologies, more and more tasks are now performed with the assistance of computers. Different industries have benefited from such technological advancement, including medical industry, where large volume of image data, capturing anatomical information of a patient, may be processed by a computer to identify anatomical structures of interest (e.g., organs, bones, blood vessels, or abnormal nodule), obtain measurements for each object of interest (e.g., dimension of a nodule growing inside an organ), and visualize relevant features (e.g., three-dimensional (3D) visualization of an abnormal nodule). FIG. 1A shows an exemplary 3D model 100 constructed for an organ (e.g., liver) based on medical data (e.g., CT scan) and various signal processing techniques. As illustrated in FIGS. 1A and 1B, the 3D model 100 characterizes not only the physical features of an organ (e.g., shape, surface, and solid body) but also internal structures of other related anatomical parts (e.g., nodules 110 growing inside of the modeled liver and blood vessels 120). Such techniques provide means to assist healthcare workers (e.g., doctors) to treat patients more effectively. For instance, physicians may use such characterizing information to perform pre-surgical planning and/or guide them during surgeries.

For example, FIG. 1C shows an example application of such techniques in a laparoscopic procedure. This setting includes a patient 135 lying on a surgical table 130 with a laparoscopic camera 140 and a surgical instrument 150 inserted into the patient's body. The surgical instrument is inserted for performing an intended surgical task and is manipulated by a surgeon based on information observed in two-dimensional video 155 with visual information captured by the laparoscopic camera 140. The laparoscopic camera 140 may be manipulated to capture a certain field of view so that the captured visual information as displayed in laparoscopic view 155 may be relied on to maneuver the surgical instrument 150 to reach an intended part of an organ and to perform the intended tasks.

While laparoscopic views may provide live 2D visualization of 3D anatomies inside a patient's body, a user needs significant experience to interpret the information presented in 2D images in order to map to 3D anatomies. FIG. 1D shows a 2D image 160-1 of a lung, where only the surface of the lung is visible. The full 3D structure of the lung as well as the internal anatomical structures therein are not visible. During a surgery, the lung may shift in position or deform in shape due to, e.g., that the patient is lying flat or breathes during the surgery. As another example, a surgeon may flip a disturb a part of the lung in order to see another part, making surrounding anatomical structures significantly change positions, shapes, or sizes. FIG. 1D may present in 2D image 160-1 with a lung in its initial position with a boundary 170. FIG. 1E is another 2D view 160-2 of the same lung at a different time, where the lung visually appears quite different when some part is flipped open or pushed away during the surgery.

Anatomical structures visible in a 2D laparoscopic image are limited due to multiple reasons. First, a laparoscopic camera has a limited field of view so that 2D images may capture only a partial view of a targeted organ. In addition, anatomical structures may extend to beneath the visible organ surface (such as blood vessels extending to internal tissue of the organ). This is illustrated in FIG. 1E, where although some blood vessels (white structures 180) are visible, they are not complete as they further extend into the organ. This is also illustrated in FIG. 1F, where two blood vessel portions (180-1 and 180-2) are visible near a tip 190 of an observed surgical tool 150. However, it is not possible to know whether the observed 180-1 and 180-2 correspond to the full structure, as either one may submerge into the organ. For these reasons, it is difficult to ascertain what is seen in 2D images corresponds to which part of the actual organ.

A surgeon often needs to identify certain blood vessels correctly during a surgery. For instance, major blood vessels may need to be clamped before cutting a target organ. To do so, a surgeon needs to identify the vessel that needs to be clamped. However, based on the deformation nature of anatomical structures in limited local and partial views of vessels, as discussed herein, it is difficult to do. A surgeon needs to mentally piece together partial information as seen in 2D images in order to guess which part of the 3D organ structure it corresponds to. This requires substantial experience. Effort has been made to register the information in 2D images with a 3D model of a target organ, various challenges remain due to nearly constant deformation of anatomical structures during the surgery.

Thus, there is a need for a solution that addresses the challenges discussed above.

### SUMMARY

The teachings disclosed herein relate to methods, systems, and programming for information management. More particularly, the present teaching relates to methods, systems, and programming related to hash table and storage management using the same.

In one example, a method, implemented on a machine having at least one processor, storage, and a communication platform capable of connecting to a network for deformable alignment. 2D vessels are automatically detected from 2D images having anatomical structures captured by a camera inserted into a patient's body and directed to a target organ. A vessel label is obtained to at least one 2D vessel. A 3D vessel structure in a 3D model for the target organ is identified based on the detected 2D vessels. The 2D vessels are aligned with 3D vessels in the 3D vessel structure so that alignment parameters for each pair may be derived and used for visualizing 3D vessels corresponding to the detected 2D vessels.

In a different example, a system is disclosed for deformable alignment, which includes a vessel structure detection unit, a vessel-based deformable alignment unit, and a vessel-based multi-window display unit. The vessel structure detection unit is provided for detecting 2D vessels from 2D images having anatomical structures captured by a camera inserted into a patient's body directed to a target organ, obtaining a vessel label for at least one 2D vessel, and identifying a 3D vessel structure in a 3D model for the target organ based on the detected 2D vessels. The vessel-based deformable alignment unit is provided for aligning detected 2D vessels with 3D vessels in the 3D vessel structure to derive alignment parameters, which may be used by the vessel-based multi-window display unit to visualize 3D vessels corresponding to the detected 2D vessels.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for deformable alignment. The information, when read by the machine, causes the machine to perform various steps. 2D vessels are automatically detected from 2D images having anatomical structures captured by a camera inserted into a patient's body and directed to a target organ. A vessel label is obtained to at least one 2D vessel. A 3D vessel structure in a 3D model for the target organ is identified based on the detected 2D vessels. The 2D vessels are aligned with 3D vessels in the 3D vessel structure so that alignment parameters for each pair may be derived and used for visualizing 3D vessels corresponding to the detected 2D vessels.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A shows an exemplary 3D model of an organ;

FIG. 1B shows that a 3D model for an organ may also models the internal anatomical structures internal or around a target organ;

FIG. 1C shows an exemplary set up of a laparoscopic procedure;

FIG. 1D shows an example partial organ visible from a laparoscopic image;

FIGS. 1E-1F show deformation of an organ and incomplete views of anatomical structures in laparoscopic images;

FIG. 2A depicts an exemplary high level system diagram of a vessel-based deformable tracking framework, in accordance with an embodiment of the present teaching;

FIG. 2B illustrates exemplary types of user input that may be leveraged to facilitate deformable alignment and tracking, in accordance with an embodiment of the present teaching;

FIG. 2C is a flowchart of an exemplary process of a vessel-based deformable tracking framework, in accordance with an embodiment of the present teaching;

FIG. 3A depicts an exemplary high level system diagram of a vessel structure detection unit, in accordance with an embodiment of the present teaching;

FIG. 3B is a flowchart of an exemplary process of a vessel structure detection unit, in accordance with an embodiment of the present teaching;

FIG. 4A depicts an exemplary high level system diagram of a user interaction interface, in accordance with an embodiment of the present teaching;

FIG. 4B is a flowchart of an exemplary process of a user interaction interface, in accordance with an embodiment of the present teaching;

FIG. 5A shows exemplary vessel structures identified near a detected specific type of surgical tool, in accordance with an embodiment of the present teaching;

FIGS. 5B-5D show user provided labels for vessels detected from 2D images, inconsistency thereof with a partial 3D vessel model localized based on the labels, and automatic correction of the labels based on the inconsistency, in accordance with an embodiment of the present teaching;

FIG. 6A depicts an exemplary high level system diagram of a vessel-based deformable alignment unit, in accordance with an embodiment of the present teaching;

FIG. 6B is a flowchart of an exemplary process of a vessel-based deformable alignment unit, in accordance with an embodiment of the present teaching;

FIG. 7 shows exemplary side-by-side visualization of vessel-based deformable alignment to enable dynamic tracking, in accordance with an embodiment of the present teaching;

FIG. 8 is an illustrative diagram of an exemplary mobile device architecture that may be used to realize a specialized system implementing the present teaching in accordance with various embodiments; and

FIG. 9 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well known methods, procedures, components, and/or system have been described at a relatively high-level, without detail, in order to avoid unnecessarily obscuring aspects of the present teachings.

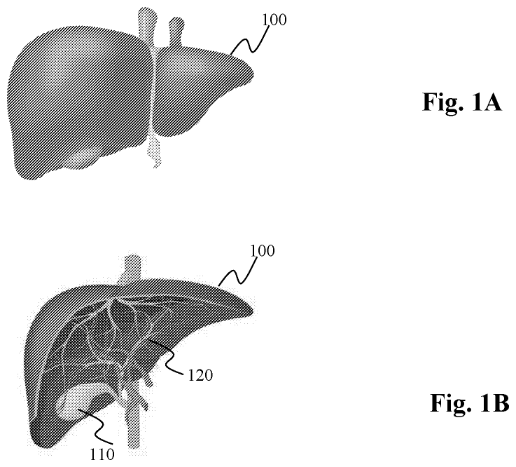

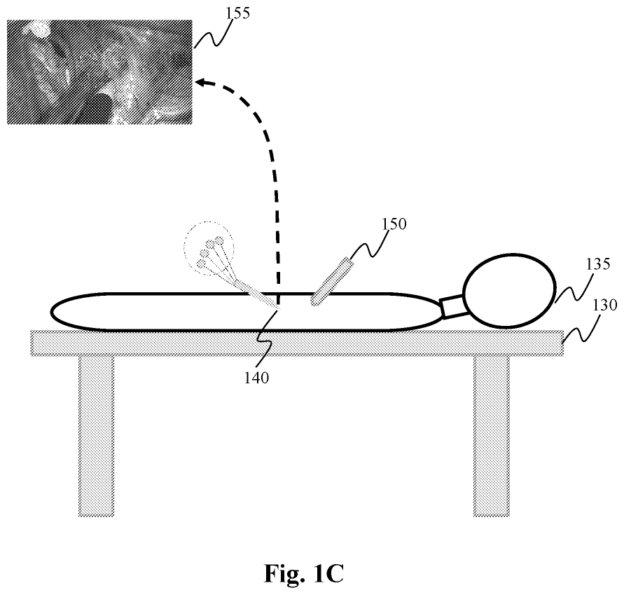

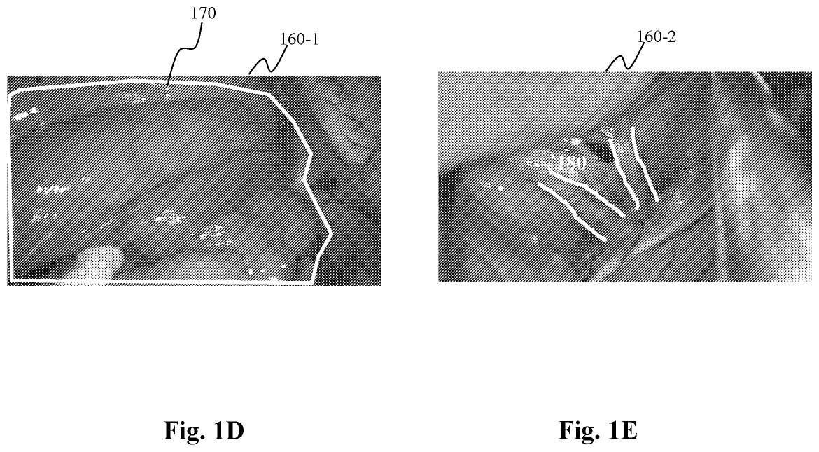

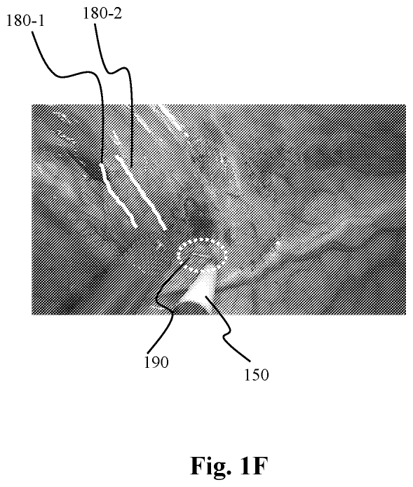

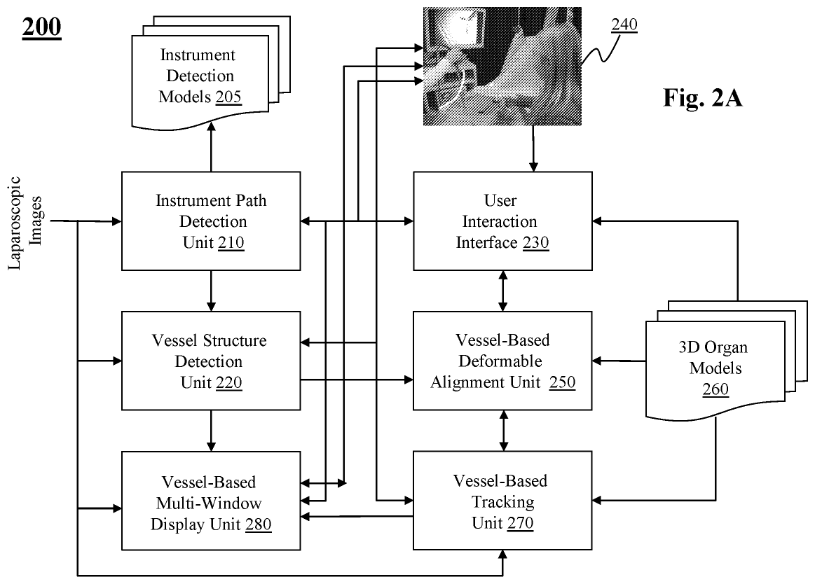

The present teaching discloses exemplary methods, systems, and implementations for aligning 2D images showing deformable anatomical structures of an organ with a 3D model for the organ. In laparoscopic surgeries, to effectively guide a user to navigate a surgical instrument to perform intended tasks, anatomical structures as seen by a laparoscopic camera are to be registered with a 3D model for the target organ. As discuss herein, due to various reasons, anatomical structures deform during a surgery, posing significant challenges in registering or aligning partial information in 2D laparoscopic images with a 3D model. The present teaching leverages partial vessels observed in 2D images as anchor anatomical structures to facilitate deformable alignment. When vessels are detected from 2D images, they may be marked as illustrated in FIG. 1F, where two blood vessels 180-1 and 180-2 are detected and marked via, e.g., centers of the vessels. Feedback from a user (e.g., a surgeon) may be received to affirm or disaffirm the marked vessels or provide estimated labels of the vessels. In some situations, such labels may correspond to the medical terms of the vessels. In other situations, such labels may be merely distinct markings when it is difficult to discern what each detected 2D vessel corresponds to an anatomically distinct vessel.

Such user provided labels may be used to localize a portion of the 3D vessel model to identify corresponding candidate 3D vessels. If a medical name of a vessel is available, the corresponding 3D vessel can be readily identified based on the label of the vessel. When the input label is merely a marking (such as A or B), the correspondences between a 2D detected vessel and a 3D vessel may be identified based on, e.g., spatial relationships formed by the 2D vessels. For example, vessel A is on the left side of vessel B and vessels A and B form a fork at some point. Such a spatial relationship among marked vessels labels may then be compared with that of the 3D vessels. If a match (either an exact of inexact match) is found on a spatial relationship, a corresponding 3D vessel structure (a part of the 3D vessel model) may be localized. In comparison, if inconsistency is detected, it may be used to facilitate automatic corrections to user estimated vessel labels. With the corrected labels of the 2D vessels detected from the laparoscopic images, correspondence between what is observed in a 2D image and the 3D model may be established so that vessels observed in laparoscopic images may be, even when deformed, registered, or aligned with vessels in the 3D vessel model.

Through alignment based on vessel spatial relations, other anatomical structures appearing in laparoscopic images, either explicitly or implicitly, may also be accordingly registered with corresponding parts in the 3D model. In some embodiments, it may be performed based on, e.g., the spatial relationships among different anatomical structures. In this manner, even though anatomical structures of a patient deform during a surgery, alignment may be handled in a more reliable manner. Such alignment scheme according to the present teaching not only represents an alternative and more reliable registration of deformable targets but also facilitates tracking, over time, the correspondences between deformable anatomical structures as observed in 2D images and that in a 3D model, providing the basis of effective navigational guidance to a surgeon during a surgery.

In some embodiments, detection of vessels present in laparoscopic images may be initiated when certain specific type of surgical tool (such as a hook) is detected. When such the specific surgical tool is detected in a surgery, it may indicate that the surgeon intends to handle vessel related tasks so that it may be inferred that both the specific surgical tool and vessels may be nearby of each other. One example is shown in FIG. 1F, where a surgical hook (190) is nearby several blood vessels (180). Thus, when the specific surgical tool is detected at some locale in 2D images, vessels may also be detected in the same vicinity.

The present teaching addresses the challenges in aligning deformable anatomical structures with corresponding parts of a 3D model via an approach as discussed herein that improves the reliability of alignment and reduces the level of difficulty, making it possible to efficiently align and track deforming anatomical parts with respect to corresponding 3D parts represented in a 3D model to enhance the ability to provide effective navigational guidance to a surgeon during a surgery.

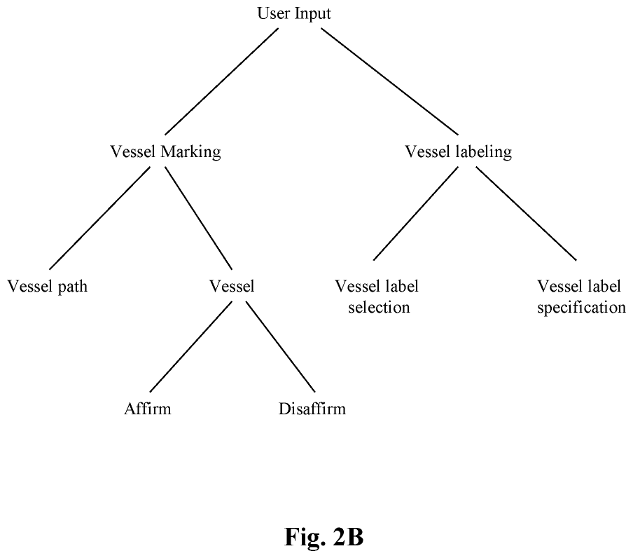

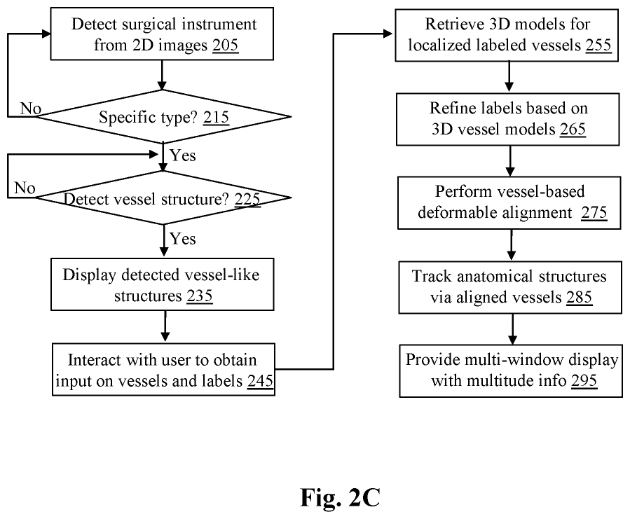

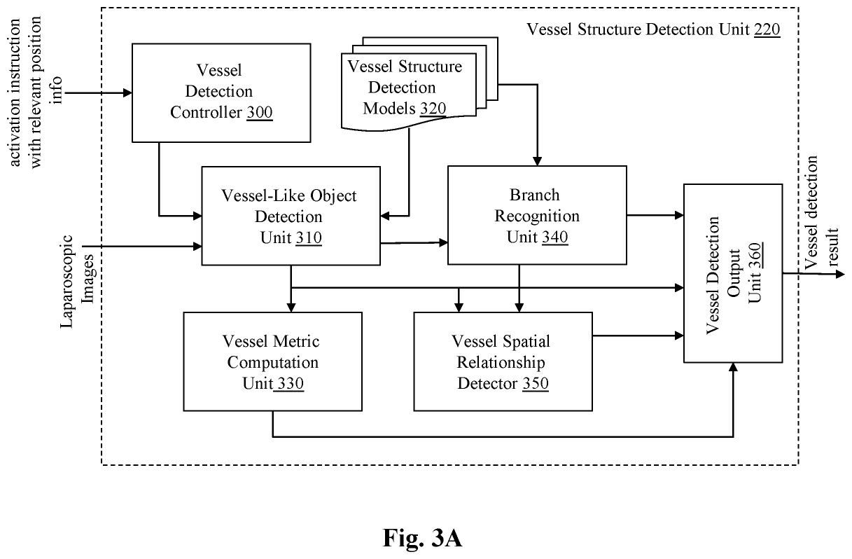

FIG. 2A depicts an exemplary high level system diagram of a vessel-based deformable tracking framework 200, in accordance with an embodiment of the present teaching. The illustrated framework 200 comprises a vessel path detection unit 210, a vessel structure detection unit 220, a user interaction interface 230, a vessel-based deformable alignment unit 250, a vessel-based tracking unit 270, and a vessel-based multi-window display unit 280. The instrument path detection unit 200 is provided for detecting and tracking, from input laparoscopic images, a surgical instrument used in a surgery and captured by a laparoscopic camera. The detection may be performed based on instrument detection models 205. The detected surgical instrument may be characterized by its positions at different times and such positions of the surgical instrument may form an instrument path or a trajectory.

In some embodiments, the instrument path detection unit 210 may be configured to detect a specific type of instrument such as a surgical hook. In detecting the specific surgical instrument, the instrument path detection unit 210 may detect not only the specific instrument but also other relevant information that may characterize the detected instrument, such as a particular part of the instrument such as the tip of a surgical hook. The position of the tip may be used to characterize the position of the instrument. A series of tip positions tracked with the movement of the hook may be used to generate an instrument path of the surgical tool.

The vessel structure detection unit 220 may be provided to recognize vessel-like structures present in laparoscopic images. As discussed herein, in some embodiments, vessel detection may be triggered when the instrument path detection unit 210 detects a specific type of surgical instrument such as a surgical hook. Positions of the surgical hook may be utilized by the vessel structure detection unit 220 to determine a region in a vicinity of each position in a 2D image to identify vessel-like structures. For example, the tip location of a detected surgical hook in a 2D image may be used by the vessel structure detection unit 220 to perform detection around the tip location. An example is shown in FIG. 1F, where two vessels 180-1 and 180-2 are detected in an area nearby a tip location 190 of a detected surgical instrument 150.

The vessel-based multi-window display unit 280 may be provided for visualize different types of information to a surgeon on a display screen (see 240) to provide visual guidance to the surgeon. The vessel-based multi-window display unit 280 may display laparoscopic images on a display screen. The vessel-based multi-window display unit 280 may also visualize signal processing results, including, e.g., tip positions of a detected surgical instrument, a movement trajectory of an instrument (detected by the instrument path detection unit 210), and/or the vessel structures appearing in the images (detected by the vessel structure detection unit 220). The vessel-based multi-window display unit 280 may also be configured to display an input provided by a user, e.g., a label specified for a detected vessel. Some signal processing results may be visualized in separate side-by-side virtual windows on the display screen. Some may be visualized by superimposing on the 2D images. For instance, the tip positions of a detected surgical hook may be superimposed on laparoscopic images to visualize the trajectory of the tool. In another example, vessel structures detected from laparoscopic images may be represented as centerlines of the vessels which are then superimposed on the laparoscopic images, as illustrated in FIGS. 1E-1F . A surgeon may specify a preferred visualization approach as called for based on application needs or personal choice.

The user interaction interface 230 is provided to facilitate a user (e.g., a surgeon or an assistant) to interact with the system 200. There may be different types of input that a user may provide to system 200. As discussed herein, the present teaching may leverage user input in the process of aligning deformable anatomical structures with corresponding parts of a target organ represented by a 3D model to make the alignment process more efficient and effective. FIG. 2B illustrates exemplary types of user input that may be leveraged to facilitate deformable alignment and tracking, in accordance with an embodiment of the present teaching. As illustrated, a user input may include an indication from a user that may affirm or disaffirm a vessel detected and visualized on a display screen. In some embodiments, a user input may also correspond to labels provided by a user for different detected vessels by, e.g., selecting vessel representation visualized on the display screen and specifying a label therefor.

As discussed herein, according to the present teaching, deformable alignment problem may be approached by leveraging vessel structures and their spatial relationship with optionally interactive labeling from a user to significantly improve the efficiency and effectiveness of aligning what is in laparoscopic images with corresponding parts of a 3D model for a target organ. The vessel-based deformable alignment unit 250 is provided to perform deformable alignment based on vessels structures automatically identified near a detected specific surgical instrument, labels for such vessel structures (estimated or obtained via interactions with a user), and a localized portion of a 3D model 260 for a target organ involved in the surgery. The alignment result from the vessel-based deformable alignment unit 250 may establish the correspondences between different anatomical structures as observed in laparoscopic images and some parts of the 3D model 260 that model these anatomical structures.

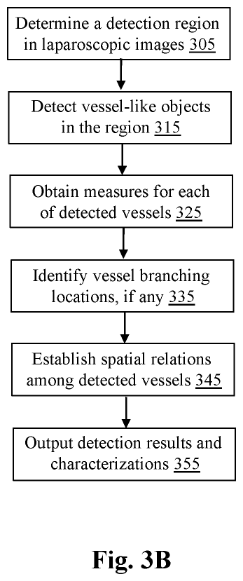

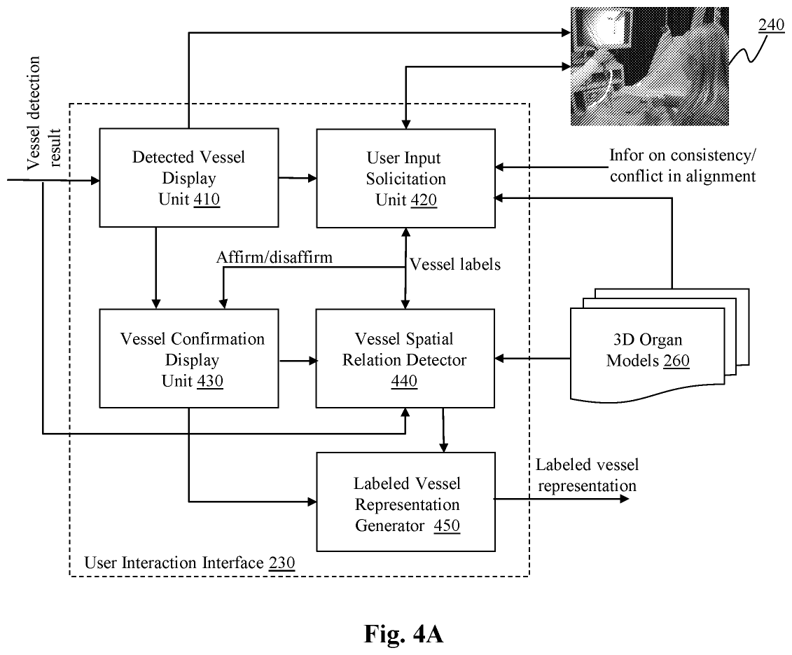

With the deformable alignment result, the dynamic correspondences between what is seen in laparoscopic images and patient's 3D anatomical structures may be tracked over time by the vessel-based tracking unit 270. During tracking, new vessels may be identified from laparoscopic images and aligned with additional parts of the 3D model in accordance with the approach as discussed herein. Such continuously tracked anatomical structures and their corresponding parts in the 3D model may also be visualized by the vessel-based multi-window display unit 280. For example, the alignment result showing dynamic correspondences may be visualized, e.g., via side-by-side views, as a visual guide to a user during a laparoscopic procedure. Details about different units (220, 230, and 250) will be provided with references to FIGS. 3A-6B .

FIG. 2C is a flowchart of an exemplary process of the vessel-based deformable tracking framework 200, in accordance with an embodiment of the present teaching. When the instrument path detection unit 210 receives 2D laparoscopic images acquired by a laparoscopic camera, it detects, at 205, surgical instrument appears in the 2D images based on instrument detection models 205. As discussed herein, to leverage vessels as an anchor in deformable alignment, vessel-like structure detection may be performed when a specific type of surgical instrument is deployed. In different surgeries, before a surgeon operates on a target organ, vessels may need to be handled appropriately. For example, some vessels may need to be pushed aside, some vessels may need to be clamped to stop the blood flow, and some vessels may need to be cut and then clamped at the ends. Different situations may require use of different surgical tools. For instance, a surgical hook may be used to separate a vessel from tissues, push it aside, or lift it so that it can be clamped. Appearance of such specific surgical tools may signal that vessels are nearby.

As discussed herein, in some embodiments, vessel detection is triggered when a specific type of surgical instrument is detected. When the instrument path detection unit 210 detects a surgical instrument, it is examined, at 215, whether the detected surgical instrument corresponds to a specific type of surgical tool (e.g., a surgical hook). If it is not the specific type of surgical tool, the instrument path detection unit 210 may continue to detect, at 205, a surgical tool appearing in the 2D images at 205. If the detected surgical tool is the specific type anticipated, the vessel structure detection unit 220 may be activated to identify vessel-like structures in 2D images. When a detected 2D structure is vessel-like, determined at 225, the detection result may be displayed, at 235, on a display screen to visualize the vessel-like structure, as illustrated in FIGS. 1E-1F, where detected vessel structures are displayed as centerlines of the vessels.

The visualized vessels detected from 2D images may be affirmed/disaffirmed or given user specified labels. The user interaction interface 230 interacts, at 245, with the user to obtain user inputs regarding the detected vessel-like structures and labels for structures that are affirmed. For example, based on user's input, some vessels may be affirmed as vessels and labeled with estimated vessel names. Such input may be estimated as what is visualized may not be complete (e.g., some nearby connected vessels may not be seen, and some detected vessels may submerge into the tissue of the organ and not visible). In some situations, the system may provide some candidate label names next to a visualized vessel-like structure so that the user may simply select one based on experience. This may be possible when a vessel is previously labeled in prior frames and the currently visualized vessel is detected via tracking the previously labeled vessel. In some implementations, the user may specify or assign a label for a detected vessel.

Based on detected vessels with labels thereof (either estimated or assigned), the vessel-based deformable alignment unit 250 localizes corresponding parts of the 3D model and retrieves, at 255, corresponding 3D vessel model(s) therefrom that correspond to the detected 2D vessels. Based on the spatial relationships among detected 2D vessels and the 3D spatial relationships among corresponding 3D vessels, the vessel-based deformable alignment unit 250 may then refine, at 265, the labels of the detected vessels based on the retrieved 3D vessel models. In this manner, the vessels detected from 2D laparoscopic images may be aligned with the 3D model for the target organ even if the vessels as appearing in 2D images are deformed. Such correspondence is used by the vessel-based deformable alignment unit 250 to align, at 275, deformed vessels detected from laparoscopic images with corresponding vessels modeled in the 3D model.

With aligned vessels between 2D and 3D, in subsequent laparoscopic image frames, the vessel-based tracking unit 270 may facilitate continuous deformable alignment by tracking, at 285, 2D vessels appearing in 2D images based on the prior vessel detection and alignment results as well as the guidance from the 3D vessel models previously aligned. The tracking result may be continually provided to the vessel-based deformable alignment unit 250 so that the alignment may be performed continuously. Such continuous detection, tracking, and alignment may result in continuous updates that may be provided to the vessel-based multi-window display unit 280 so that it can provide, at 295, on-the-fly update on information related to the detected 2D vessels, the aligned 3D vessels models, or fused views of projecting 3D vessels models on 2D vessels when appropriate (e.g., when the deformation is not substantial).

FIG. 3A depicts an exemplary high level system diagram of the vessel structure detection unit 220, in accordance with an embodiment of the present teaching. The vessel structure detection unit 220 is provided for detecting vessel-like structures. In some embodiments, the activation of the vessel structure detection unit 220 may be triggered when a specific defined surgical instrument is detected from laparoscopic images. In this illustrated embodiment, the vessel structure detection unit 220 comprises a vessel detection controller 300, a vessel-like object detection unit 310, a vessel metric computation unit 330, a branch recognition unit 340, a vessel spatial relationship detector 350, and a vessel detection output unit 360. The vessel detection controller 300 is provided to control when to activate the detection of vessel-like structures. In some embodiments, it is based on an activation instruction received from the instrument path detection unit 210 when a specific surgical instrument is detected from 2D images. Such an activation instruction may be provided with additional information used to facilitate vessel detection at relevant location(s). For instance, the tip position of a specific surgical instrument detected from a 2D image may be provided with the activation instruction so that vessel detection may be carried out in a region in the 2D image around the tip of the specific surgical tool. Upon receiving the activation instruction, the vessel detection controller 300 may invoke the vessel-like object detection unit 310 with, e.g., information on the position of the specific surgical instrument.

The vessel-like object detection unit 310 takes laparoscopic images as input and recognize vessel-like object(s) in the images. In some situation, it may operate to detect across an entire image plane of each frame. In other situation, the detection may be directed to a region around a given position on the image plane. The operational mode may be controlled by the vessel detection controller 300. In the latter mode, it may be activated by the vessel detection controller 300 with information indicative of a position in the image plane, representing the location of the specific surgical tool. In this configuration, the vessel-like object detection unit 310 may determine a region in laparoscopic images around the location of the surgical tool so that vessel detection can be performed within the determined region based on, e.g., vessel structure detection models 320. This may improve the relevancy as well as efficiency of the detection process. The size of the region for detection may be determined based on application needs, which in turn may be specified according to different types of surgeries.

For detected vessel-like structures, some associated features may also be determined, including, e.g., location, diameter, centerline, or curvature of each vessel, branch point if any, as well as the spatial relationship among different vessels. Such features may facilitate subsequent tasks. For instance, the size and curvature of a vessel may be used to identify candidate labels that may be associated thereto. The spatial relationship among different vessel-like structures may also provide cues as to estimated labels. The vessel metric computation unit 330 may be provided for computing measurements associated with each detected vessel-like structure. The branch recognition unit 340 is provided for determining whether detected vessel-like structures form any branch(es) according to some criteria. The vessel spatial relationship detector 350 may be provided for identifying spatial relationships among detected vessel-like structures. For example, structure A resides on left of structure B in 2D images and A and B are the only neighbor to the other, etc. Such recognized spatial relationship between/among different vessel-like structures may be used to estimate their respective labels.

The outputs from the vessel-like object detection unit 310 (2D vessel-like structures), the vessel metric computation unit 330 (e.g., for determining characterizing measures for each detected structure), the branch recognition unit 340 (e.g., for determining whether branch point exist with respect to which detected vessels), and the vessel spatial relationship detector 350 (e.g., for detecting how detected structures spatially relate to each other) may then be provided to the vessel detection output unit 360, which may then combine these detection results to generate an output that characterizes the vessel-like structures present in some region in an 2D image plane. As discussed herein, such detection results may then be used by the vessel-based multi-window display unit 280 so that the detection result may be visualized to provide a basis for soliciting inputs from a user.

FIG. 3B is a flowchart of an exemplary process of the vessel structure detection unit 220, in accordance with an embodiment of the present teaching. This flowchart describes the process of vessel-like structure detection once activated. As discussed herein, in some embodiments, the activation may be based on an instruction created when a specific surgical instrument is detected from laparoscopic images. Once activated, a detection region in an image plane may be determined, at 305, based on a position related to the specific surgical tool detected. The vessel-like object detection unit 310 detects, at 315, vessel-like structures, e.g., within the detection region. The detected vessel-like structures are sent to different units to, e.g., obtain characterizing measures of each vessel-like structure at 325, identify vessel branches at 335, and identify spatial relationships between/among different detected vessels at 345. Such vessel detection results are then combined to produce an output at 355.

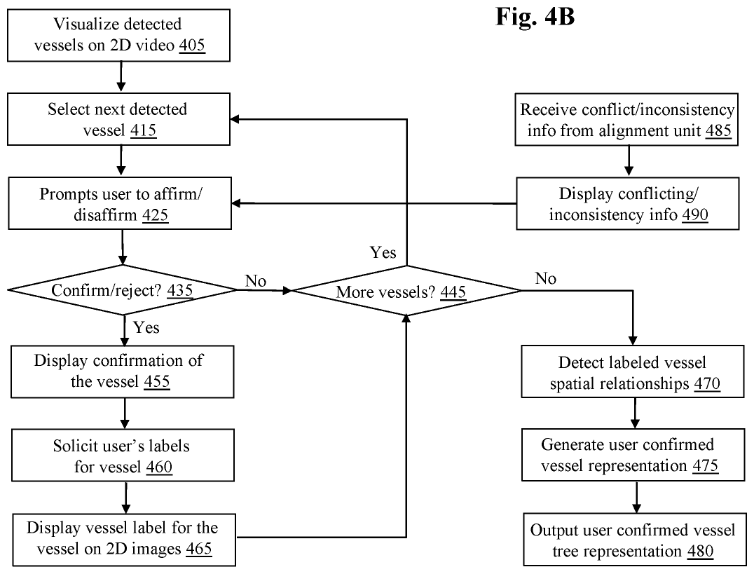

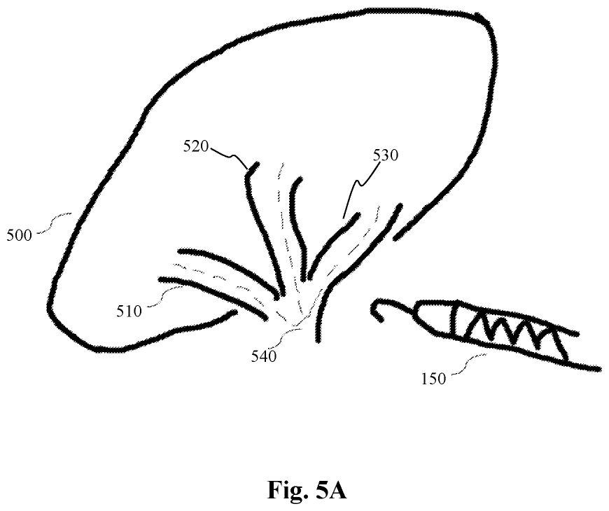

FIG. 4A depicts an exemplary high level system diagram of the user interaction interface 230, in accordance with an embodiment of the present teaching. As discussed herein, to reduce the complexity of deformable alignment in laparoscopic procedures in order to provide meaningful in-surgery guidance to a user, in some embodiments, the present teaching may leverage both vessel-like structures detected from 2D images as anchors as well as user input to enhance the effectiveness and efficiency in deformable alignment. The user interaction interface 230 is provided to leverage user input via human-machine interactions. In this illustrated embodiment, the user interaction interface 230 comprises a detected vessel display unit 410, a user input solicitation unit 420, a vessel confirmation display unit 430, a vessel spatial relation detector 440, and a labeled vessel representation generator 450. The user interaction interface 230 takes the vessel-like structures and relevant information thereof from vessel structure detection unit 220 and output confirmed vessels with estimated vessel labels.

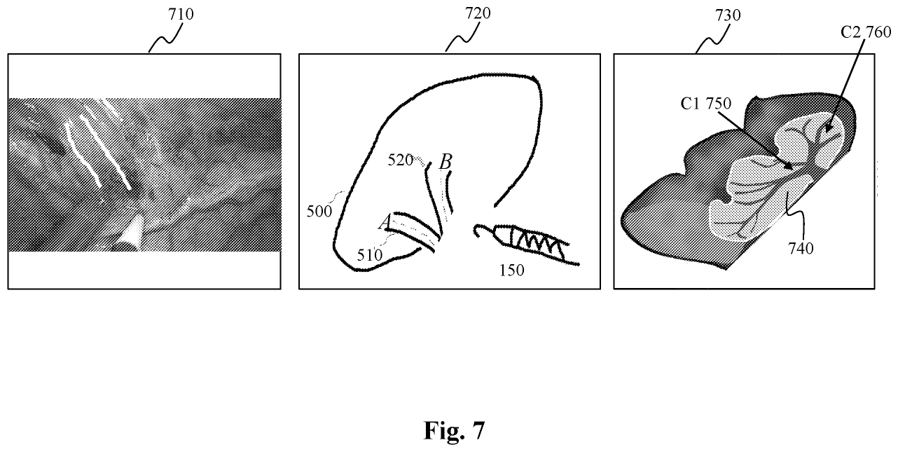

The detected vessel display unit 410 takes the vessel detection result and render them on the display screen. As discussed herein, the visualization may be in a side-by-side image displays (one example is shown in FIG. 7) or by superimposing the detected vessel-like structures on 2D laparoscopic images (as shown in FIGS. 1E-1F). The visualization allows a user to see what is detected around a surgical instrument. The detected vessel display unit 410 may activate the user input solicitation unit 420 to initiate interaction with the user via display screen 240. With respect to each detected vessel-like structure, the user input solicitation unit 420 may request the user to affirm or disaffirm the vessel detection results. When the user's input to affirm or disaffirm vessels is received, the user input solicitation unit 420 sends the user input to the vessel confirmation display unit 430 which may then update the visualization of the detected vessels incorporating the user's input. In some embodiments, the disaffirmed vessels may be removed from the visualization while the affirmed vessels may remain in the visualization display screen. In some embodiments, the disaffirmed vessels may be marked differently (e.g., using a different color or intensity) to distinguish from the affirmed vessels. The affirmed vessels may then be sent to the vessel spatial relation detector 440 and the labeled vessel representation generator 450.

The vessel spatial relation detector 440 may be provided for identifying, based on labeled affirmed vessels, candidate corresponding 3D vessels represented in the 3D model 260. To do so, labels for the affirmed vessels may first be estimated, e.g., by a user via interactions with the user input solicitation unit 420. For each of the affirmed vessels, a user may provide an estimated label. When all affirmed vessels are assigned estimated labels, they are used by the vessel spatial relation detector 440 to establish the spatial relationship between/among labeled vessels which is then used to identify, from the 3D model 260, candidate corresponding 3D vessels that form similar or the same spatial relationships.

In some embodiments, the vessel labels may initially be automatically estimated so that a user may be requested to affirm, disaffirm, or provide an alternative vessel label. In this situation, the vessel detection result may be provided to the vessel spatial relation detector 440 so that it may rely on the spatial relationship recognized between/among detected 2D vessels to identify candidate 3D corresponding vessels from 3D model 260 that form similar or the same spatial relationships. By doing so, it allows the vessel spatial relation detector 440 to estimate vessel labels based on that of the candidate 3D corresponding vessels for the 2D vessels. Because 2D vessels detected may not be complete or each 2D vessel may not be visible fully (e.g., due to occlusion), the candidate vessel labels initially estimated in this manner may not be correct. However, they may be used as a starting point for soliciting a user's input with respect to the estimated vessels labels so that the estimated labels may be refined based on user's input. To do so, the user solicitation unit 420 may operate to display the initially estimated vessel labels and then solicit the user's affirmation or rejection or a new specified label.

The user input regarding vessel labels (either user provided alternative vessel label or feedback on the automatically generated estimated vessel labels) create updated spatial relationship of the 2D vessels, which may then be used by the vessel spatial relation detector 440 to again identifies updated candidate corresponding 3D vessels from the 3D organ models 260. The labeled vessel representation generator 450 may be provided to obtaining a representation for the labeled vessels based on the affirmed 2D vessels (detected from laparoscopic images) as well as the candidate corresponding 3D vessels from the 3D model 260. The representation include labeled vessels in both 2D and 3D and is output to the vessel-based deformable alignment unit 250 for carrying out deformable alignment based on the estimated correspondences between 3D and 3D vessels.

In some embodiments, such estimated correspondences between 2D and 3D vessels may still include incorrect correspondence, which may cause inconsistency or conflict during alignment. Such inconsistency/conflict may be fed back (from the vessel-based deformable alignment unit 250) to the user input solicitation unit 420 to inform the user of such inconsistency/conflict as the basis for seeking for further user input such as a corrected label for a vessel in question. The updated label(s) may again be provided to the vessel spatial relation detector 440 to update the spatial relationship and then accordingly identify updated candidate 3D vessels that correspond to the 2D vessels. The newly identified 2D/3D vessel correspondence may then be generated accordingly by the labeled vessel representation generator 450 and output to the vessel-based deformable alignment unit 250 to perform further alignment to remedy the previously detected inconsistency/conflict. The deformable alignment according to the present teaching may involve such back and forth until the inconsistency/conflict are resolved.

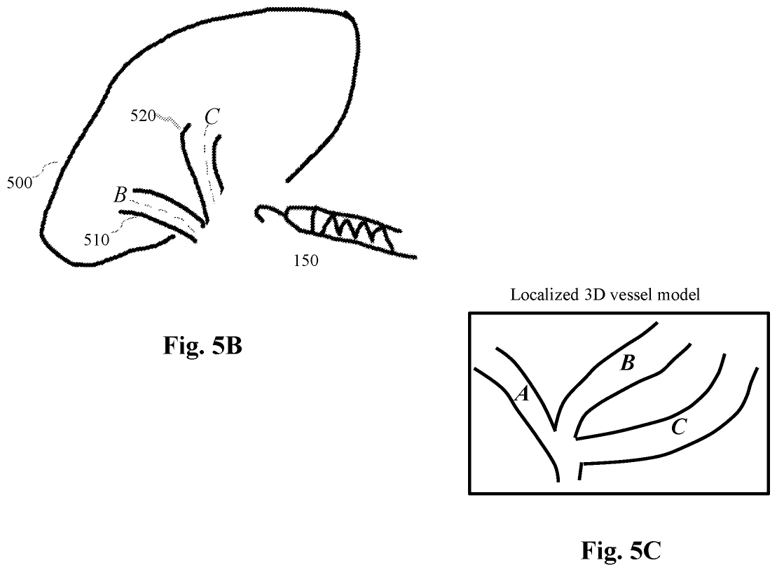

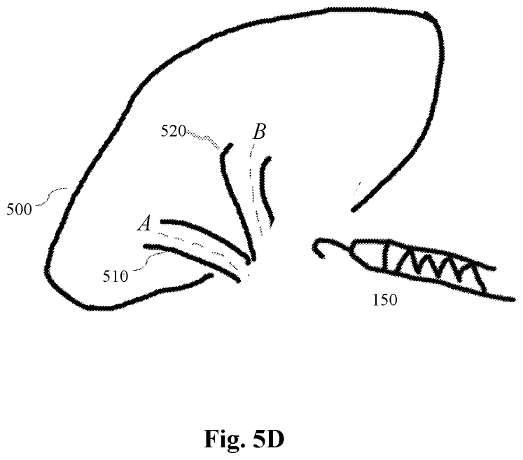

FIGS. 5A-5D illustrate an example situation with inconsistency detected during deformable alignment and correction on vessel labels accordingly, in accordance with an embodiment of the present teaching. FIG. 5A shows exemplary vessel structures (510-530) in an organ 500. As seen, a specific surgical instrument (e.g., a hook) 150 may be detected when approaching organ 500 and it triggers the detection of vessels nearby the detected specific surgical instrument. Assume that an anatomical model includes three vessels, i.e., vessel 510, vessel 520, and vessel 530 in a close range, as shown in FIG. 5A, and they are respectively modeled in a 3D model as corresponding 3D vessels as A, B, and C, as shown in FIG. 5C, i.e., vessel 510 has a label A, vessel 520 has a label B, and vessel 530 has a label C.

In a laparoscopic procedure, when specific surgical instrument 150 is detected from laparoscopic images, it triggers the detection of 2D vessels in a region in the same images near the location of the detected tool 150. FIG. 5B shows the vessels detection result, which shows that only vessels 510 and 520 are detected. Vessel 530 is not detected due to, e.g., any of different reasons. For instance, it could be due to the image quality (vessel 530 is simply not visible); it could be that vessel 530 resides beneath the visible surface of the organ 500; it could also be that vessel 530 is currently occluded by something else in front of it. As only two vessels are detected, although a user may provide input to affirm the two detected vessels, ambiguity and, hence, uncertainty, exists in assigning labels to the detected vessels. In the example shown in FIG. 5B, the user assigns label B to vessel 510 and label C to vessel 520. That is, the user assumes that what is not detected is vessel A, 2D vessel 510 corresponds to 3D vessel B, and 2D vessel 520 corresponds to 3D vessel C.

The initial labels so provided (either via automatic estimation or user specified) may cause inconsistency/conflict in deformable alignment. According to initial labels B and C for two 2D vessels, candidate corresponding 3D vessels B and C (as shown in FIG. 5C) may be identified. However, according to the 3D model for the localized vessel tree, there are three branches A, B, and C and they form a certain spatial relationship, i.e., B is in the middle, A is on the left of B, and C is on the right of B. This required spatial relationship may not be present or supported in 2D images, e.g., if there is evidence to support that vessel 510 (currently labeled as B) has no left neighbor or that vessel 520 has a right neighbor. This causes ambiguity in deformable alignment as it cannot be certain whether 2D 510/520 pair corresponds to 3D A/B pair or 3D B/C pair. Such inconsistency or conflict between what is observed in 2D and what is modeled in 3D models may be identified in aligning what is detected in 2D with what is modeled in 3D models and may be fed back to the user to seek information to disambiguate. For example, in this example, when the user is informed of the inconsistency, the user may relabel the detected vessels 510 and 520 with labels A and B, respectively, as shown in FIG. 5D. In some situations, the ambiguity may not be resolved until additional information is available, e.g., more vessels are detected and one of them may reside on right of vessel 520 and form, with vessels 510 and 520, the same spatial relationship as that of 3D vessels A/B/C.

FIG. 4B is a flowchart of an exemplary process of the user interaction interface 230, in accordance with an embodiment of the present teaching. When the detected vessel display unit 410 receives the vessel detection results, it visualizes, at 405, the detected 2D vessels. Upon visualization, the user input solicitation unit 420 may then interact with the user to seek user input (e.g., affirmation or disaffirmation or labels thereof) with respect to each of the detected 2D vessels. When a vessel is selected for processing at 415, the user input solicitation unit 420 prompts, at 425, a user for affirming or disaffirming the vessel. When the user's input is received, it is determined, at 435, whether the user affirms or disaffirms the vessel. In this case, the vessel confirmation display unit 430 displays, at 455, information on the display screen indicative the fact that the vessel detected is accepted. When the current vessel is affirmed by the user, the user input solicitation unit may further solicit, at 460, the user's input on the label of the vessel. When the user provides an input indicating the label to be assigned to the vessel, the user input solicitation unit 420 forwards the user's specified label to the vessel confirmation display unit 430 so that it may display, at 465, the user's specified label for the vessel on the display screen.

Upon the update of the visualization of a confirmed vessel (updated vessel visualization and label display) at 465 or if the vessel is rejected by the user, determined at 435, the process proceeds to 445, where it is checked to see whether there is any additional detected 2D vessel that needs to be processed. If so, the process moves to step 415 to select the next vessel to repeat the same process. Otherwise, the process proceeds to construct spatial relationship between/among the confirmed vessels by sending confirmed vessels and their labels to the vessel spatial relation detector 440, which detects, at 470, the labeled spatial relationship(s) between/among confirmed vessels and provides (together with the confirmed labeled vessels from the vessel confirmation display unit 430) the detected spatial relationship(s) to the labeled vessel representation generator 450, which then generates, at 475, labeled vessel representation and outputs the same at 480 to the vessel-based deformable alignment unit 250.

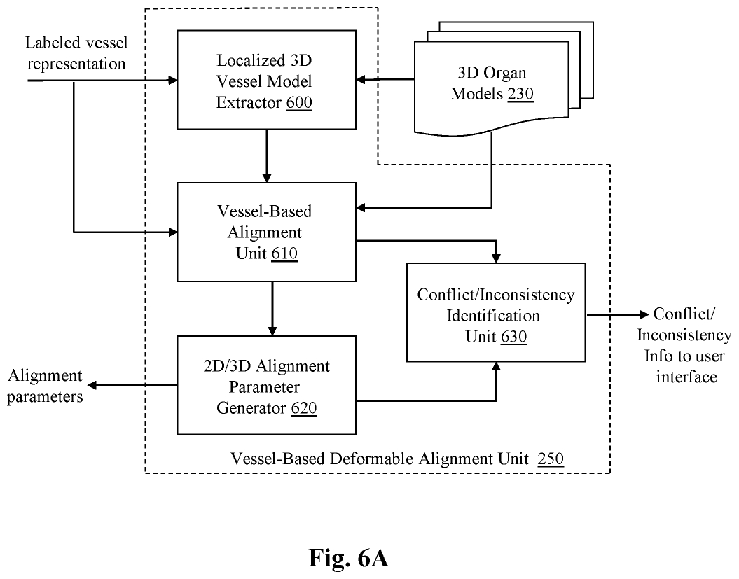

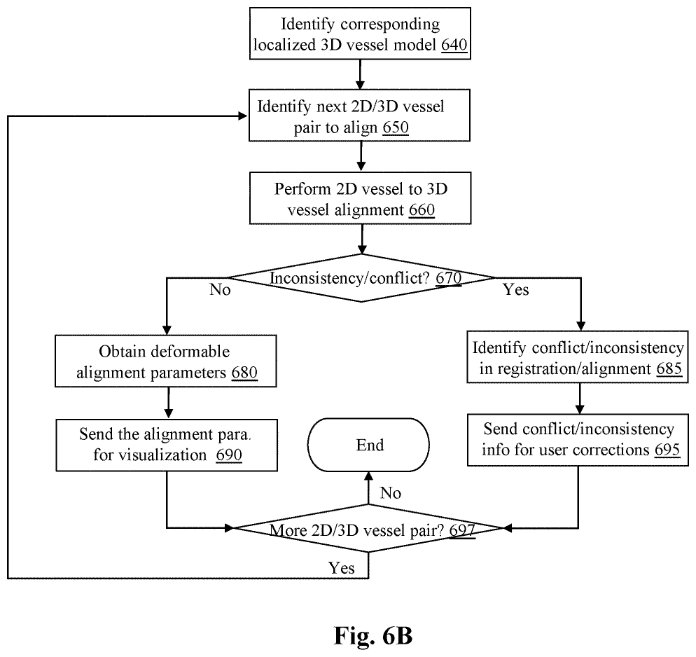

FIG. 6A depicts an exemplary high level system diagram of the vessel-based deformable alignment unit 250, in accordance with an embodiment of the present teaching. As discussed herein, to register deformed objects appearing in 2D laparoscopic images to corresponding anatomical parts modeled by a 3D model 260 for a target organ, the present teaching leverages vessels detected from 2D images as well as user's input as to vessel and labels thereof to identify candidate corresponding 3D vessels in the 3D model. The vessel-based deformable alignment unit 250 is provided to achieve the registration based on labeled vessels obtained based on laparoscopic images and comprises a localized 3D vessel model extractor 600, a vessel-based alignment unit 610, a 2D/3D alignment parameter generator 620, and a conflict/inconsistency identification unit 630. The localized 3D vessel model extractor 600 is provided for identifying a part of the 3D model associated with 3D vessels in the target organ that corresponds to the labeled 2D vessels detected from 2D images. For example, as illustrated in FIG. 5D, the detected vessels 510 and 520 have labels A and B. The 3D model may include a part for modeling a vessel structure with labels A, B, and C. In this case, although the part of the 3D vessel model with A, B, C vessels is not a complete match with 2D vessel detection result (because C is missing), the spatial relationship between vessel A and vessel B is consistent between 2D and 3D results. Thus, the localized 3D vessel model extractor 600 may select the part of the 3D model involving vessel labels A and B as a corresponding vessel structure of the 2D vessel detection result.

In some embodiments, the localized part of the 3D model may also be identified via automatic means. The detected 2D vessel-like structures may form a certain spatial relationship. For example, two vessel-like structures may form a fork, one may be on the left of the other two, etc. Such spatial configuration may be used to identify 3D vessels represented in the 3D model that have a same or similar spatial configuration among different vessels. A matching may be performed to identify localized structures in the 3D model with vessels structured in a way that is similar to that of the detected 2D vessel-like structures. For instance, if it is detected that three vessels meet at the same position to form a three-way branching point, such a unique spatial relation may be used to identify a localized vessel tree in the 3D model that includes a branching point that is at least a three-way construct. Such a spatial relation based approach may be applied in different situations, e.g., when user assigned vessel label is not available or when a real time situation cannot afford the time to solicit users' input.

Once the corresponding part of the 3D model is selected, the vessel-based alignment unit 610 is provided to align each of the 2D vessels detected with a corresponding 3D vessel in the selected 3D vessel model. Due to various reasons, in some embodiments, the alignment may be carried out one vessel at a time. For example, to align a 2D vessel with its corresponding 3D vessel in a way so that the projection of the 3D vessel yields the 2D vessel as it appears in laparoscopic images, a transformation may need to be applied to the 3D vessel. Due to deformation, the transformation needed for each vessel may differ and may be individually identified but the spatial relation between/among different vessels in both 2D and 3D spaces remains the same. As discussed herein, if the 2D detection has ambiguities, inconsistency/conflict between 2D detection result and 3D vessel model may exist, such as the case shown in FIGS. 5B and 5C. The conflict/inconsistency identification unit 630 is provided to identify such inconsistency/conflict. The identified inconsistency/conflict may be fed back to the user interaction interface 230 to address as discussed above so that the labels for 2D vessels may be adjusted.

If no inconsistency/conflict is identified during the alignment, the 2D/3D registration parameter generator 620 may generate deformable alignment parameters for each pair of corresponding 2D/3D vessels. Such alignment parameters may be used to align each 2D/3D pair of corresponding vessels and may be used to project a 3D vessel to the plane of 2D laparoscopic images. Such deformable alignment parameters may also be provided to the vessel-based tracking unit 270 to facilitate continuous tracking the correspondences between 2D laparoscopic images and the 3D models for the target organ, which is the basis for provide effective guidance to the user in the laparoscopic procedure.

FIG. 6B is a flowchart of an exemplary process of the vessel-based deformable alignment unit 250, in accordance with an embodiment of the present teaching. When the localized 3D vessel model extractor 600 receives representation of labeled 2D vessel (from the user interaction interface 230), it analyzes the labels and their spatial relationship to identify, at 640, a part of the 3D model 230 (localized 3D vessel model) that corresponds to the labeled 2D vessel representation. The identification may be performed by, e.g., matching the spatial relations between/among 2D vessels and that of 3D vessels. As discussed herein, the alignment may be performed on a vessel-by-vessel basis. Based on the identified localized 3D vessel model, each of the 2D vessels may be paired with a corresponding 3D vessel from the model for individual alignment. At 650, the next 2D/3D vessel pair is selected and the 2D extracted vessel is aligned with, at 660, a 3D vessel from the localized 3D vessel model. During the alignment process, if there is no inconsistency/conflict detected, determined at 670, the 2D/3D alignment parameter generator 620 generates, at 680, deformable registration parameters based on the alignment result and sends, at 690, the alignment parameters to, e.g., the vessel-based tracking unit 270. If inconsistency/conflict is present during alignment, determined at 670, the conflict/inconsistency identification unit 630 generates, at 685, feedback relating to the inconsistency/conflict and sends, at 695, the feedback to the user interaction interface 230 so that the user interaction interface 230 may seek a correction in labeling the relevant vessel from the user. The vessel-by-vessel alignment process continues until all 2D/3D vessel pairs have been aligned, determined at 697.

As discussed herein, in a laparoscopic procedure, a surgeon may rely on what is shown in laparoscopic images as a guide to determine how to manipulate a surgical instrument to perform an intended task on a target organ inside of a patient's body. In recent years, a 3D model previously created for the target organ and associated anatomical structures prior to the surgery may be used to further enhance the ability to assist the surgeon. For example, projecting a relevant portion of a 3D model onto the laparoscopic images assists a surgeon by providing a rendered 3D view of what is in front of a surgical instrument. Projecting 3D structures onto 2D images may become more difficult when relevant anatomical structures are deformed. Even when deformable alignment is successful, projecting an entire 3D structure such as a vessel structure onto laparoscopic images is difficult as the relative spatial positions of different vessels may have been changed. In some embodiments, based on the alignment parameters derived individually for each 2D/3D vessel pairs according to the present teaching, each individual 3D vessel from the localized 3D vessel model may be projected onto the 2D laparoscopic images separately based on its own set of alignment parameters. In this manner, 3D vessels identified via deformable alignment may still be projected onto the laparoscopic images even when the 2D vessels are deformed.

FIG. 7 shows an exemplary side-by-side visualization 700 of information that is made possible due to vessel-based deformable alignment, in accordance with an embodiment of the present teaching. As illustrated in FIG. 7, in a multi-window visualization theme, there may be side-by-side display windows 710, 720, and 730, each of which is for visualizing different types of information. In this example, display window 710 is for displaying laparoscopic video information captured by a laparoscopic camera with individually superimposed 3D vessels each of which is projected based on its separately derived alignment parameters. Display window 720 may be provided to show what is detected from the laparoscopic images and the surgeon's input on the detection results. For example, as shown in FIG. 7, there is a detected surgical instrument 150 at its location, 2D vessels 510 and 520 detected nearby the surgical instrument 150 at their relative locations, as well as vessel labels (A to vessel 510 and B to vessel 520, either automatically estimated for surgeon's approval or specified by the surgeon). Display window 730 may be provided to visualize the localized 3D vessel model that is identified based on the vessel structures detected from laparoscopic images.

In this example, a sub-vessel model 740 is extracted from the 3D model 260 as relating to the 2D vessels 510 and 520 and it is visualized in window 730. In this visualized sub-vessel tree 740, there may be different parts that may be considered as candidate correspondence to detected 2D vessels, including the part C1 750 and the part C2 760. As discussed herein, such candidates may be identified via matching the spatial relationships between two sets of 2D/3D vessels and are marked as candidates (C1 and C2) so that a surgeon may proceed to select one based on experience. These side-by-side display windows may be visualized by the vessel-based multi-window display unit 280 based on information from other functional units and then controlled by the user interaction interface 230 as the basis to interact with a surgeon seeking confirmation of the detection result, specification of vessel labels assigned thereto, and approval of the corresponding 3D sub-vessel tree if needed. The side-by-side display windows provide a surgeon with different types of information to allow the surgeon to get an improved sense of what he/she is facing. Such enriched information derived based on the deformable alignment according to the present teaching may provide effective assistance to a surgeon during a laparoscopic procedure.

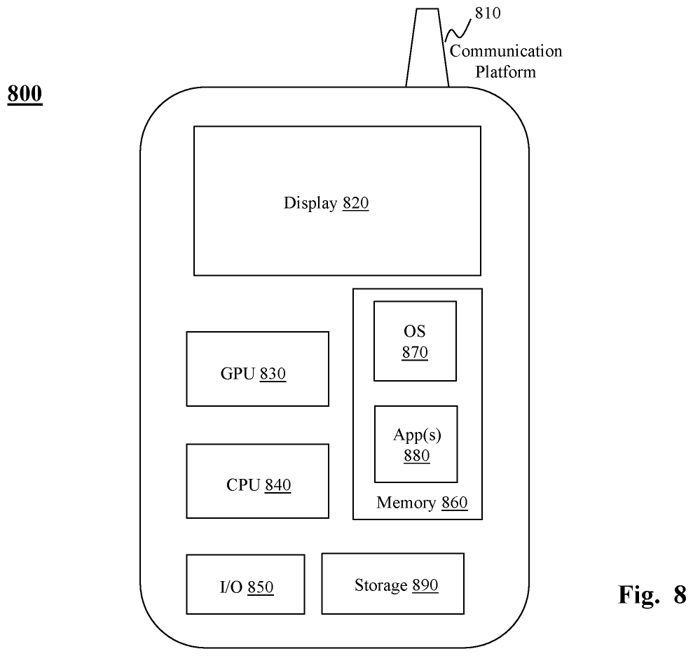

FIG. 8 is an illustrative diagram of an exemplary mobile device architecture that may be used to realize a specialized system implementing the present teaching in accordance with various embodiments. In this example, the user device on which the present teaching may be implemented corresponds to a mobile device 800, including, but not limited to, a smart phone, a tablet, a music player, a handled gaming console, a global positioning system (GPS) receiver, and a wearable computing device, or in any other form factor. Mobile device 800 may include one or more central processing units (“CPUs”) 840, one or more graphic processing units (“GPUs”) 830, a display 820, a memory 860, a communication platform 810, such as a wireless communication module, storage 890, and one or more input/output (I/O) devices 850. Any other suitable component, including but not limited to a system bus or a controller (not shown), may also be included in the mobile device 800. As shown in FIG. 8, a mobile operating system 870 (e.g., iOS, Android, Windows Phone, etc.), and one or more applications 880 may be loaded into memory 860 from storage 890 in order to be executed by the CPU 840. The applications 880 may include a user interface or any other suitable mobile apps for information analytics and management according to the present teaching on, at least partially, the mobile device 800. User interactions, if any, may be achieved via the I/O devices 850 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result the drawings should be self-explanatory.

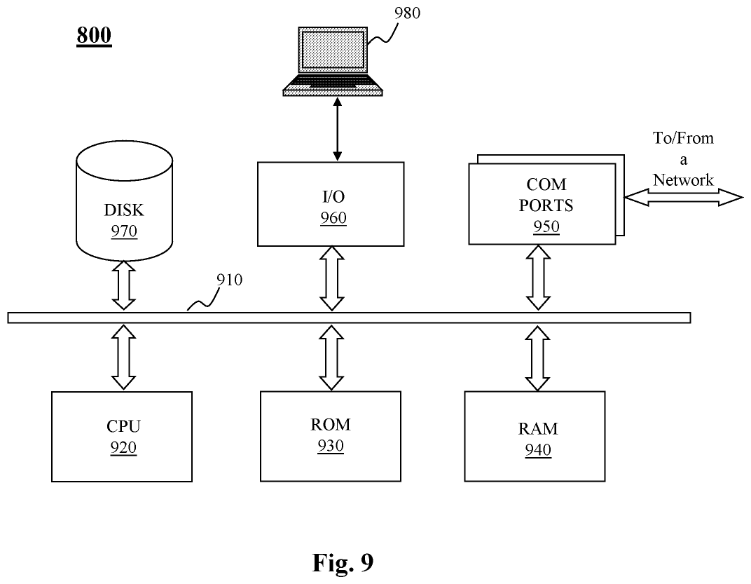

FIG. 9 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special purpose computer. Both can be used to implement a specialized system for the present teaching. This computer 800 may be used to implement any component or aspect of the framework as disclosed herein. For example, the information analytical and management method and system as disclosed herein may be implemented on a computer such as computer 900, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 900, for example, includes COM ports 950 connected to and from a network connected thereto to facilitate data communications. Computer 900 also includes a central processing unit (CPU) 920, in the form of one or more processors, for executing program instructions. The exemplary computer platform includes an internal communication bus 910, program storage and data storage of different forms (e.g., disk 970, read only memory (ROM) 930, or random-access memory (RAM) 940), for various data files to be processed and/or communicated by computer 900, as well as possibly program instructions to be executed by CPU 920. Computer 900 also includes an I/O component 960, supporting input/output flows between the computer and other components therein such as user interface elements 980. Computer 900 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as a main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method implemented on at least one processor, a memory, and a communication platform, comprising:
receiving two-dimensional (2D) images from a camera inserted into a patient's body directed to a target organ, wherein the 2D images capture anatomical structures of the patient;
automatically detecting one or more 2D vessels from the 2D images;
assigning a vessel label to each of the one or more 2D vessels;
identifying a three-dimensional (3D) vessel structure from a 3D model for the target organ based on the labeled one or more 2D vessels, wherein the 3D vessel structure represents the one or more 2D vessels in a 3D space with the corresponding labels;
aligning each of the one or more 2D vessels with a corresponding 3D vessel in the 3D vessel structure to derive alignment parameters; and
visualizing 3D vessels corresponding to the one or more 2D vessels based on the alignment parameters.

2. The method of claim 1, wherein
at least some of the one or more 2D vessels are deformed; and
the camera captures a surgical instrument inserted into the patient's body for performing an intended action in relation to the target organ.

3. The method of claim 2, wherein the step of detecting comprises:
automatically identifying a pre-determined type of surgical instrument in the 2D images and an associated 2D position;
extracting the one or more 2D vessels in a region in the 2D images near the 2D position;
generating a representation for each of the one or more 2D vessels; and
determining at least one 2D spatial relationship among the one or more 2D vessels based on their representations.

4. The method of claim 1, wherein the step of assigning a vessel label to each of the one or more 2D vessels comprises:
with respect to each of the one or more 2D vessels,
visualizing the 2D vessel based on a representation of the 2D vessel,
receiving an input including a vessel label for the 2D vessel when the 2D vessel is confirmed, and
associating the vessel label with the 2D vessel.

5. The method of claim 4, wherein the vessel label is obtained by:
detecting a 2D spatial relationship formed by the one or more 2D vessels;
matching the 2D spatial relationship with that of vessels represented in the 3D model to locate a 3D vessel structure with one or more 3D vessels forming a spatial relationship similar to the 2D spatial relationship;
identifying a 3D vessel in the 3D vessel structure that corresponds to the 2D vessel; and
retrieving a vessel label associated with the identified 3D vessel as the vessel label of the 2D vessel.

6. The method of claim 1, wherein the step of identifying the 3D vessel structure comprises:
obtaining at least one vessel label associated with some of the one or more 2D vessels, wherein each of the at least one vessel label represents a medical name of one of the some 2D vessels;
locating the 3D vessel structure in the 3D model that includes 3D vessels having vessel labels corresponding to the at least one vessel label of the some 2D vessels.

7. The method of claim 1, wherein the step of aligning comprises:
determining a transformation used to project the corresponding 3D vessel so that the projection yields an appearance similar to that of the 2D vessel;
obtaining alignment parameters based on the transformation, and
sending the alignment parameters for visualizing the corresponding 3D vessel in place of the 2D vessel.

8. A machine-readable and non-transitory medium having information recorded thereon, wherein the information, when read by the machine, causes the machine to perform the following steps:
receiving two-dimensional (2D) images from a camera inserted into a patient's body directed to a target organ, wherein the 2D images capture anatomical structures of the patient;
automatically detecting one or more 2D vessels from the 2D images;
assigning a vessel label to each of the one or more 2D vessels;
identifying a three-dimensional (3D) vessel structure from a 3D model for the target organ based on the labeled one or more 2D vessels, wherein the 3D vessel structure represents the one or more 2D vessels in a 3D space with the corresponding labels;
aligning each of the one or more 2D vessels with a corresponding 3D vessel in the 3D vessel structure to derive alignment parameters; and
visualizing 3D vessels corresponding to the one or more 2D vessels based on the alignment parameters.

9. The medium of claim 8, wherein
at least some of the one or more 2D vessels are deformed; and
the camera captures a surgical instrument inserted into the patient's body for performing an intended action in relation to the target organ.

10. The medium of claim 9, wherein the step of detecting comprises:
automatically identifying a pre-determined type of surgical instrument in the 2D images and an associated 2D position;
extracting the one or more 2D vessels in a region in the 2D images near the 2D position;
generating a representation for each of the one or more 2D vessels; and
determining at least one 2D spatial relationship among the one or more 2D vessels based on their representations.

11. The medium of claim 8, wherein the step of assigning a vessel label to each of the one or more 2D vessels comprises:
with respect to each of the one or more 2D vessels,
visualizing the 2D vessel based on a representation of the 2D vessel,
receiving an input including a vessel label for the 2D vessel when the 2D vessel is confirmed, and
associating the vessel label with the 2D vessel.

12. The medium of claim 11, wherein the vessel label is obtained by:
detecting a 2D spatial relationship formed by the one or more 2D vessels;
matching the 2D spatial relationship with that of vessels represented in the 3D model to locate a 3D vessel structure with one or more 3D vessels forming a spatial relationship similar to the 2D spatial relationship;
identifying a 3D vessel in the 3D vessel structure that corresponds to the 2D vessel; and
retrieving a vessel label associated with the identified 3D vessel as the vessel label of the 2D vessel.

13. The medium of claim 8, wherein the step of identifying the 3D vessel structure comprises:
obtaining at least one vessel label associated with some of the one or more 2D vessels, wherein each of the at least one vessel label represents a medical name of one of the some 2D vessels;
locating the 3D vessel structure in the 3D model that includes 3D vessels having vessel labels corresponding to the at least one vessel label of the some 2D vessels.

14. The medium of claim 8, wherein the step of aligning comprises:
determining a transformation used to project the corresponding 3D vessel so that the projection yields an appearance similar to that of the 2D vessel;
obtaining alignment parameters based on the transformation, and
sending the alignment parameters for visualizing the corresponding 3D vessel in place of the 2D vessel.

15. A system, comprising:
a vessel structure detection unit implemented by a processor and configured for
receiving two-dimensional (2D) images from a camera inserted into a patient's body directed to a target organ, wherein the 2D images capture anatomical structures of the patient,
automatically detecting one or more 2D vessels from the 2D images, and
obtaining a vessel label for each of the one or more 2D vessels a vessel-based deformable alignment unit implemented by a processor and configured for
identifying a three-dimensional (3D) vessel structure from a 3D model for the target organ based on the labeled one or more 2D vessels, wherein the 3D vessel structure represents the one or more 2D vessels in a 3D space with the corresponding labels, and
aligning each of the one or more 2D vessels with a corresponding 3D vessel in the 3D vessel structure to derive alignment parameters; and
a vessel-based multi-window display unit implemented by a processor and configured for visualizing 3D vessels corresponding to the one or more 2D vessels based on the alignment parameters.

16. The system of claim 15, wherein
at least some of the one or more 2D vessels are deformed; and
the camera captures a surgical instrument inserted into the patient's body for performing an intended action in relation to the target organ.

17. The system of claim 16, wherein the step of detecting comprises:
automatically identifying a pre-determined type of surgical instrument in the 2D images and an associated 2D position;
extracting the one or more 2D vessels in a region in the 2D images near the 2D position;
generating a representation for each of the one or more 2D vessels; and
determining at least one 2D spatial relationship among the one or more 2D vessels based on their representations.

18. The system of claim 15, wherein the step of assigning a vessel label to each of the one or more 2D vessels comprises:
with respect to each of the one or more 2D vessels,
visualizing the 2D vessel based on a representation of the 2D vessel,
receiving an input including a vessel label for the 2D vessel when the 2D vessel is confirmed, and
associating the vessel label with the 2D vessel.

19. The system of claim 18, wherein the vessel label is obtained by:
detecting a 2D spatial relationship formed by the one or more 2D vessels;
matching the 2D spatial relationship with that of vessels represented in the 3D model to locate a 3D vessel structure with one or more 3D vessels forming a spatial relationship similar to the 2D spatial relationship;
identifying a 3D vessel in the 3D vessel structure that corresponds to the 2D vessel; and
retrieving a vessel label associated with the identified 3D vessel as the vessel label of the 2D vessel.

20. The system of claim 15, wherein the step of identifying the 3D vessel structure comprises:
obtaining at least one vessel label associated with some of the one or more 2D vessels, wherein each of the at least one vessel label represents a medical name of one of the some 2D vessels;
locating the 3D vessel structure in the 3D model that includes 3D vessels having vessel labels corresponding to the at least one vessel label of the some 2D vessels.

21. The system of claim 15, wherein the step of aligning comprises:
determining a transformation used to project the corresponding 3D vessel so that the projection yields an appearance similar to that of the 2D vessel;
obtaining alignment parameters based on the transformation, and
sending the alignment parameters for visualizing the corresponding 3D vessel in place of the 2D vessel.
